(19) United States
(12) Patent Application Publication
(10) Pub. No.: US 2009/0248080 A1
(43) Pub. Date: Oct. 1, 2009

(54) ALIGNMENT MARKING FOR SPINAL RODS

(75) Inventors: Bryan S. WILCOX, Collierville, TN (US); Rodney R. BALLARD, Lakeland, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/055,378

(22) Filed: Mar. 26, 2008

(51) Int. Cl. A61B 17/58; C23C 8/00; B24B 1/00

(52) U.S. Cl. 606/261; 148/240; 451/38

(57) ABSTRACT

Embodiments of methods for marking a spinal rod to aid medical personnel in placement of the spinal rod in a medical patient are disclosed. In certain embodiments, a spinal rod is anodized a first color. Next, a portion of the anodized color is removed from the spinal rod to form a marking. In other embodiments, the anodized spinal rod is anodized a second time to better differentiate the marking from the remainder of the spinal rod.

The present disclosure relates to processes used in orthopedic surgical procedures. Specifically, the present disclosure relates to a process that can be used to permanently mark one or more lines on a spinal rod.

Often a surgeon inserts a spinal rod in a medical patient to correct and stabilize damage or malformation of bones, especially the spine. It is beneficial to the surgeon if an identifying marker is placed on the spinal rod. This identifying marker can help the surgeon in contouring or shaping the spinal rod to correspond with a proper alignment of one or more vertebra in a medical patient. This mark or line is a reference by which a surgeon can bend the spinal rod to a particular contour. Additionally, the surgeon can measure the curvature of the spinal rod by measuring the line and taking dimensions from the line. The line can also help the surgeon during implantation of the rod in one or more vertebra to aid the surgeon in correcting the pathology as was intended. The proper contour or shape of the spinal rod maintains the alignment of vertebrae after implantation of the spinal rod in the medical patient.

Several techniques and systems have been developed for marking a spinal rod that is to be placed in one or more vertebrae. Among them are systems that etch a line in the surface material of the spinal rod. However, in many cases etching the spinal rod creates a localized, weakened spot on the spinal rod. Over time applied load to the spinal rod weakens the etched spot to fatigue the spinal rod which can lead to failure of the spinal rod and to potentially another surgery for the medical patient in which it was implanted. Therefore a need exists for another process of marking a spinal rod.

### SUMMARY

Among other things, a method is disclosed including anodizing a metal (e.g. titanium) spinal rod to form a first color on a surface of the spinal rod, and blasting the rod with a fluid stream to remove a substantially straight line of the first color on the surface to expose the metal spinal rod. The fluid stream may include a mixture of sand particles and air or glass beads and air. A second anodizing of the rod can be performed to form a second color on the surface of the rod to better differentiate said straight line from the remainder of the rod. The first color may be blue, and the second color may be purple or bronze. A second line, e.g. one offset from the first line by about 180 degrees, can be exposed by further blasting the rod. The rod may be attached to one or more vertebrae in a medical patient, and/or to at least one other orthopedic implant. The rod may be bent to correspond with a curvature of one or more vertebrae of a medical patient. The method may further include forming an identification code on a portion of the rod.

A method is also disclosed that includes anodizing a spinal rod (e.g. a titanium rod) to form a first color on a surface of the rod, removing a portion of the colored surface from the rod, and anodizing the rod to form a second color on the surface of the spinal rod. The removing act may include removing one or more lines of the colored surface from the rod, and a first line may be offset from a second line by about 180 degrees. The removing act may include bead blasting the rod with a mixture of air and glass beads, sandblasting the rod with a mixture of air and sand particles, or it may use a stream of compressed air. The method can include bending the rod to a desired curvature of one or more vertebrae.

Also disclosed is orthopedic medical apparatus including an anodized spinal rod having a first end separated from a second end by one or more sidewalls, with the sidewall(s) connecting the first end to the second end. At least one of the sidewall(s) defines a marking, wherein the marking was formed by removing a portion of the anodization. Each of at least two sidewalls may define a marking, and the markings may be offset from each other by approximately 180 degrees. There may be six sidewalls that form a hexagonal cross-sectional shape. The rod may be bent to a sagittal alignment of one or more vertebrae in a medical patient.

### BRIEF DESCRIPTION OF THE DRAWINGS

FIG. 1 is a side view of one embodiment of a marked spinal rod.

FIG. 2 is an end view of the embodiment of the spinal rod shown in FIG. 1 viewed in the direction of the arrows.

FIG. 3 is a side view of one embodiment of an unmarked spinal rod being anodized.

FIG. 4 is a perspective view of one embodiment of the anodized spinal rod from FIG. 3 being sandblasted.

FIG. 5 is a side view of one embodiment of a marked spinal rod with three lines.

FIG. 6 is a side view of one embodiment of a marked spinal rod.

FIG. 7 is a perspective view of one embodiment of the spinal rod as shown in FIG. 6 being implanted in a medical patient.

FIG. 8 is a flow chart indicating embodiments of marking a spinal rod, contouring a spinal rod, and implanting the spinal rod in a medical patient.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein being contemplated, as would normally occur to one skilled in the art to which the disclosure relates.

Among other things, there are disclosed methods and system for marking a spinal rod, such as a titanium support rod. Marking the spinal rod aids a medical professional by providing a reference or guide such that the professional can more precisely shape or contour the spinal rod to desired dimensions for insertion in a medical patient to ensure proper alignment of damaged vertebrae. Before surgery, a surgeon can use x-rays and the marking on the spinal rod to bend or shape the spinal rod to the proper curvature to correct the alignment of the vertebrae. During surgery, a surgeon can use the marking on the spinal rod to implant the spinal rod in a precise location in a medical patient. Post-surgery, the surgeon can use the marking to determine the stresses on the vertebrae and surrounding tissue by measuring the location of the marking relative to the corrected vertebrae. Thus, the method and the rod marking help ensure the surgeon maintains proper sagittal alignment at least while contouring the rod and during implantation to ensure he or she has corrected the patient's pathology as was intended.

To form the marking on the spinal rod at least two acts or steps are performed on the spinal rod. First, the spinal rod is anodized to oxidize the surface of the spinal rod to form a first color on the surface of the spinal rod. Next, the spinal rod is sandblasted to remove a portion of the anodized first color to expose the non-anodized portion of the spinal rod. This exposed portion is a mark and often this mark is one or more lines. In some instances, the spinal rod is anodized a second time to oxidize the mark to a first color and further oxidize the remainder of the spinal rod to form a second color to better differentiate the mark from the remainder of the spinal rod. No introduction of any potentially harmful coloring agents is necessary. Marking the spinal rod in this manner does not damage the spinal rod and does not erode a localized area of the surface of the spinal rod to create a weak spot in spinal rod which can reduce the fatigue life of the spinal rod and potentially lead to a subsequent surgery for a medical patient in which the spinal rod is implanted. Marking the spinal rod in this manner also ensures the marking remains on the spinal rod while the spinal rod is contoured or shaped either pre-surgery or during surgery, and it remains with the rod in vivo, not flaking away from the rod. It has been found that the disclosed methods are better ways to provide lines or other markings on a rod than other previously-known processes.

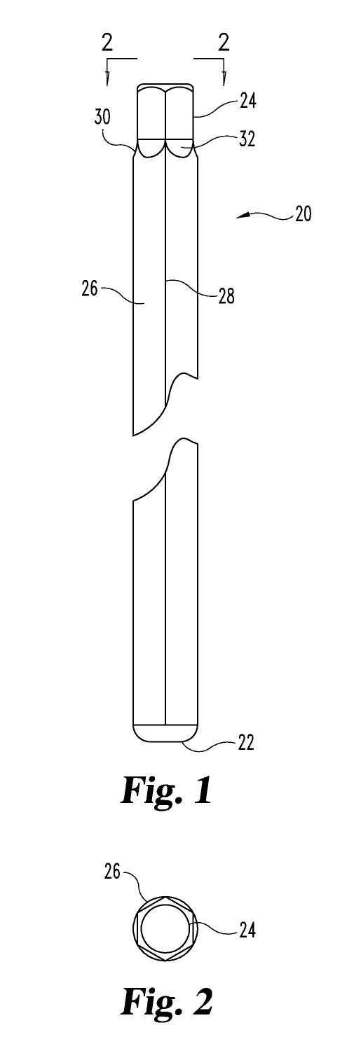

Referring generally to FIGS. 1 and 2, there is shown an embodiment of a marked spinal rod 20. Spinal rod 20 includes a first end 22 opposite a second end 24 and a sidewall 26 connecting first end 22 to second end 24. First end 22 is a semi-circular shape; however, in other embodiments, first end 22 is shaped differently such as rectangular, trapezoidal, or hexagonal. Second end 24 has a hexagonal cross-sectional shape; however, in other embodiments, second end 24 has a circular, rectangular, or trapezoidal cross-sectional shape, to name a few shapes. In the illustrated embodiment, sidewall 26 forms a circular cross-sectional shape. In other embodiments, sidewall 26 can have a different cross-sectional shape such as square, hexagonal, or some other shape.

In the illustrated embodiment, sidewall 26 includes a marking 28. In other embodiments, sidewall 26 includes a plurality of markings 28. In those embodiments with a plurality of markings 28, each marking 28 can be the same shape or each marking 28 can be shaped differently from each other. As illustrated, marking 28 is substantially a straight line. In other forms, marking 28 can be a curved line, or another shape such as a series of circles, a series of rectangles, a cross-hatch pattern, or another type of pattern or shape. In one embodiment, sidewall 26 includes two or more markings 28 with each marking 28 offset from the other marking 28 by 180 degrees. That level of offset allows the marking to be viewed from either side of the rod, so that the surgeon need not be concerned about which side of the rod is up during placement. In another embodiment, sidewall 26 includes two or more markings 28 with each marking 28 offset from the other marking by a measurement less than 180 degrees. In yet another embodiment, sidewall 26 includes two or more markings 28 with each marking 28 offset from another marking 28 by a different measurement. Such offsets permit easy viewing of torsional bending or stress, in addition to the ease of placement noted above.

In the illustrated embodiment, sidewall 26 includes a narrower tapered portion 30 adjacent second end 24. Tapered portion 30 is subdivided into six sections 32. Each of the six sections 32 connects second end 24 to sidewall 26 such that each section 32 is angled or tapered from second end 24 to the thicker portion of sidewall 26. In the illustrated embodiment, each section 32 forms a half-circle. In another embodiment, semi-circular section 32 has a radius of approximately 3 millimeters. In other embodiments, tapered portion 20 can be subdivided into any number of sections 32 or form a continuous surface from second end 24 to the thicker portion of sidewall 26. Additionally, in these other embodiments, sections 32 can be other shapes such as triangular, rectangular, or trapezoidal, to name a few.

Spinal rod 20 is made of titanium, in one particular embodiment. Other embodiments could use rods of other biocompatible metals, such as stainless steel, titanium alloys, nickel-titanium alloys, nitinol, and chrome alloy.

In one embodiment, spinal rod 20 has a length of approximately 500 millimeters and a diameter of approximately 5.5 millimeters. First end 22 forms a substantially circular end with a flat portion in the center and a rounded edge having a radius of approximately 1.5 millimeters. Second end 24 has a hexagonal cross-sectional shape. Second end 24 has a length of approximately 4.75 millimeters and a cross-sectional width between opposing sides of the hexagon of approximately 4.7 millimeters.

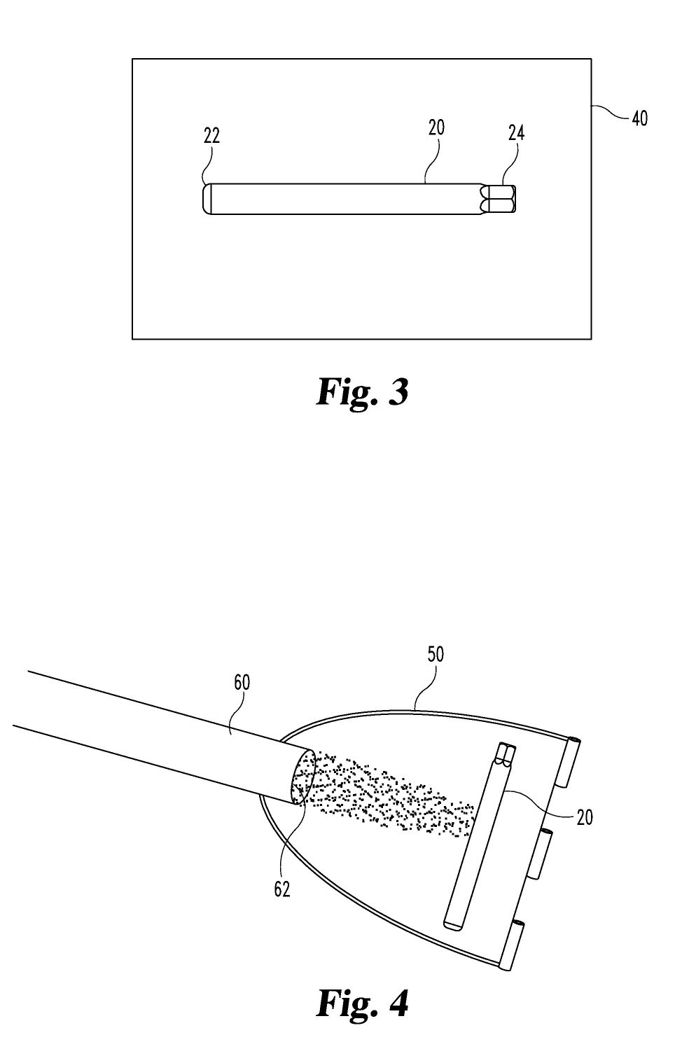

Referring generally to FIGS. 3, 4, and 5, there are shown embodiments of a system for designating the spinal rod 20 with marking 28. First, illustrated in FIG. 3, an unmarked spinal rod 20 is positioned in an anodizing device 40 that anodizes the surface of spinal rod 20. In one embodiment, spinal rod 20 is made of titanium, and after anodizing spinal rod 20 a layer of titanium dioxide is formed on the surface. Spinal rod 20 is anodized such that the oxidized surface of spinal rod 20 has a color different from the initial color (generally gray) of the rod. The color formed on the surface of spinal rod 20 is dependent mainly on the thickness of the oxide. Some factors determining the thickness of the oxide are the amount of anodizing voltage and length of time of application of the voltage. One color found to be preferential for spinal rod 20 is blue. Spinal rod 20 oxidized to a blue color was found to better distinguish a lighter colored marking 28. SAE standards for titanium anodizing are found in AMS 2487 and AMS 2488, incorporated herein by reference.

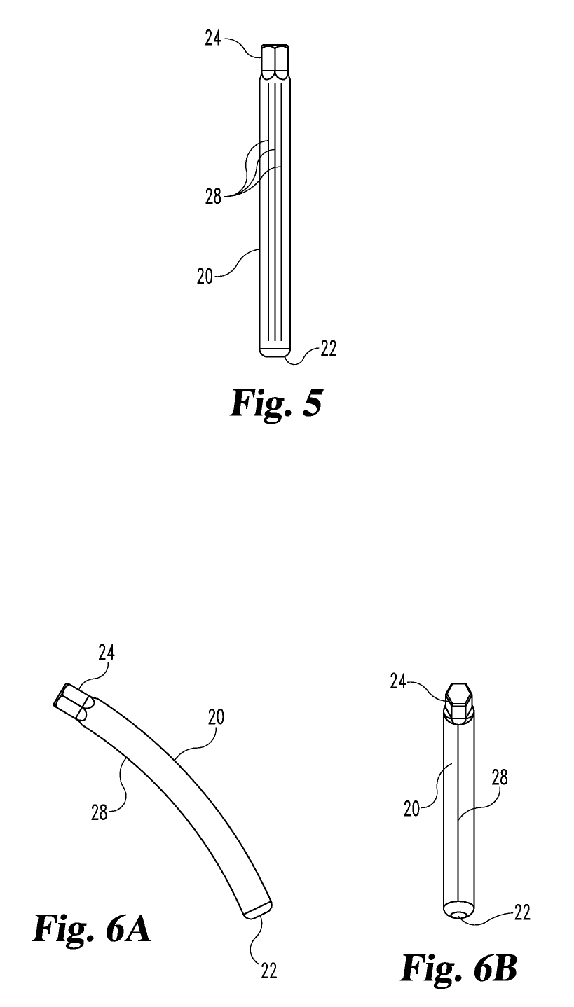

Next, illustrated in FIG. 4, anodized spinal rod 20 is positioned in a fixture 50 (top portion of fixture 50 removed to show spinal rod 20) with a discharge end 62 of a sandblasting device 60 inserted in fixture 50. In this embodiment, fixture 50 is a clamshell fixture that encloses all of spinal rod 20 and discharge end 62. At least a part of the rod 20 is within such a fixture and exposed to discharge end 62. Fixture 50 includes a top portion 52 and a bottom portion 54 connected together by a hinge 56. In other embodiments, fixture 50 is configured differently. Sandblasting device 60 forces a fluid stream of solid particles suspended in air through discharge end 62 to hit anodized spinal rod 20. Some materials used for the solid particles include sand, glass beads, metal pieces, powdered abrasives of various grades, powdered slag, and copper slag, to name a few. The repeated hitting of spinal rod 20 with the solid particles from sandblasting device 60 wears away the anodized surface of spinal rod 20 to expose the metal color of spinal rod 20 and form marking(s) 28. Thus, with the surface for spinal rod 20 anodized to a different base color, sandblasting creates a line or figure of contrast in the color (e.g. gray) of the original metal. Essentially a negative is produced, with the marking(s) 28 in the original metal color, and surround surface of a different color. One or more markings 28 can be formed on spinal rod 20, as desired. Illustrated in FIG. 5 is one embodiment of spinal rod 20 with three markings 28.

In some embodiments, the anodized spinal rod 20 with one or more markings is anodized a second time. In this embodiment, anodized spinal rod 20 is positioned in anodizing device 40 and re-anodized such that the surface is oxidized and a different, second color appears on the surface. Additionally, marking(s) 28 are oxidized to a new color. Thus, in this embodiment the surface of rod 20 is of one color, produced by first and second anodizing treatments, and marking(s) 28 are of another color produced only by the second anodizing treatment, the color of the first anodizing treatment having been removed along marking(s) 28. Such second colors include purple, bronze, or some other color darker than the first color. The re-anodizing with or to another color can provide even better distinction than with one anodization and subsequent removal of a line or other figure of that anodization. A second anodizing of spinal rod 20 occurs generally as described above. In some medical procedures a surgeon will want marking 28 better differentiated from the remainder of spinal rod 20. In these procedures, spinal rod 20 is anodized a second time such that the first color of marking 28 is distinguished from the second color of the remainder of spinal rod 20.

Illustrated in FIG. 6, spinal rod 20 is bent or contoured to a desired curvature for implantation in a medical patient to correct and/or align damaged vertebrae to a proper alignment. Marking 28 assists a medical professional by forming a reference line from which the professional can bend spinal rod 20 and measure the curvature of spinal rod 20 to ensure spinal rod 20 is accurately shaped. Additionally, a medical professional can align spinal rod 20 with marking 28 along the vertebrae in an x-ray thereby minimizing time during surgery of the medical patient since the spinal rod 20 is accurately contoured and ready for insertion into the patient.

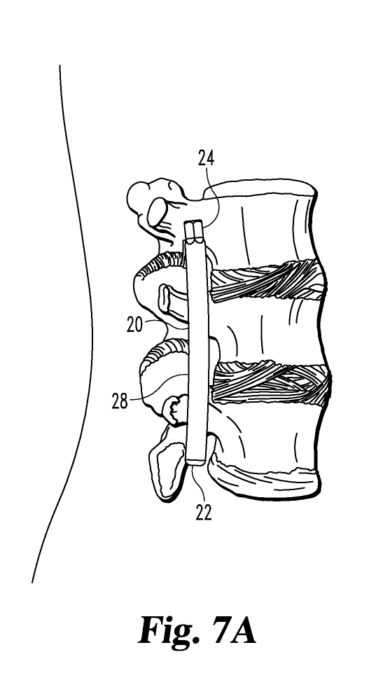

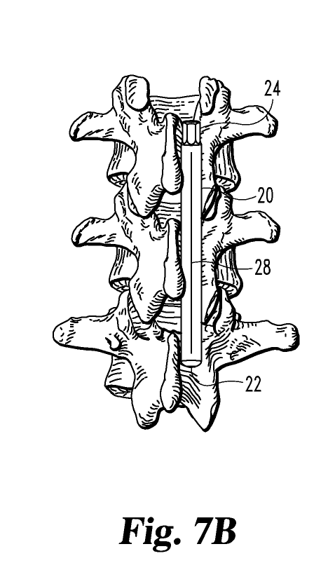

FIG. 7 illustrates spinal rod 20 inserted in a medical patient. Marking 28 assists a surgeon in correctly aligning vertebrae during a medical procedure by acting as a reference or guide that aids a surgeon to accurately position spinal rod 20 next to the appropriate vertebrae. Spinal rod 20 can be attached to other orthopedic devices and implants more easily with marking 28 forming a guide in aligning spinal rod 20 with the devices and implants.

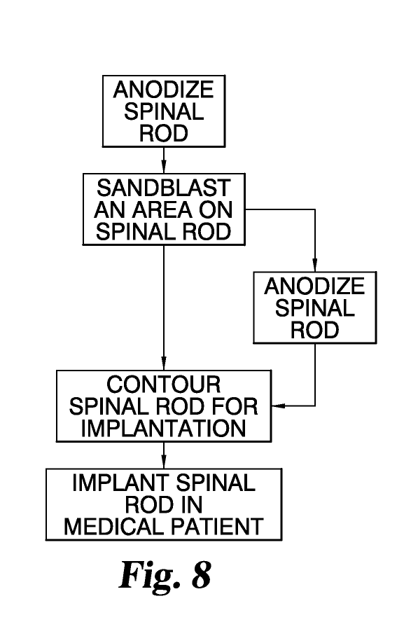

Referring to FIG. 8, there is shown a flow chart of an embodiment of forming marking 28 on spinal rod 20, contouring spinal rod 20, and implanting spinal rod 20. These embodiments have been discussed in detail above. As indicated, this embodiment reflects forming marking 28 by anodizing spinal rod 20 to oxidize the surface of spinal rod 20 and sandblasting an area on spinal rod 20 to remove a portion of the oxidized surface on spinal rod 20. In some embodiments, spinal rod 20 is anodized a second time as indicated in the flow chart. In some forms, spinal rod 20 is contoured for implantation in a medical patient. Lastly, spinal rod 20 is implanted in a medical patient.

While the disclosure has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A method comprising:
anodizing a metal spinal rod to colorize a surface of the spinal rod with a first color; and
blasting said metal spinal rod with a fluid stream to remove a substantially straight line of the first color on the surface to expose the metal spinal rod.

2. The method of claim 1, wherein said fluid stream includes a mixture of sand particles and air.

3. The method of claim 1, wherein said fluid stream includes a mixture of glass beads and air.

4. The method of claim 1, further comprising anodizing said spinal rod a second time to form a second color along at least a portion of said line to further differentiate said line from the remainder of the spinal rod.

5. The method of claim 4, wherein said anodizing to colorize with a first color is performed at a first voltage, and said anodizing a second time is performed at a second voltage, said first voltage being higher than said second voltage.

6. The method of claim 4, wherein said anodizing a second time has no effect on said first color.

7. The method of claim 1, further comprising blasting said spinal rod to expose a second line offset from the first line by about 180 degrees.

8. The method of claim 1, further comprising attaching said spinal rod to one or more vertebrae in a medical patient.

9. The method of claim 8, further comprising attaching said spinal rod to at least one other orthopedic implant.

10. The method of claim 1, further comprising bending the spinal rod to correspond with a curvature of one or more vertebrae of a medical patient.

11. The method of claim 1, further comprising forming an identification code on a portion of the spinal rod.

12. The method of claim 1, wherein said spinal rod is made of titanium.

13. A method comprising:
anodizing a spinal rod to form a first color on a surface of the spinal rod;
removing a portion of the colored surface from the spinal rod; and
anodizing the spinal rod to form a second color on the surface of the spinal rod.

14. The method of claim 13, further comprising bending said spinal rod to a desired curvature of one or more vertebrae.

15. The method of claim 13, wherein said removing act includes removing one or more lines of the colored surface from the spinal rod.

16. The method of claim 15, further comprising offsetting a first line from a second line by about 180 degrees.

17. The method of claim 13, wherein said removing act includes bead blasting said spinal rod with a mixture of air and glass beads.

18. The method of claim 13, wherein said removing act includes sandblasting said spinal rod with a mixture of air and sand particles.

19. The method of claim 13, wherein said removing act includes using a stream of compressed air.

20. An orthopedic medical apparatus comprising:
an anodized spinal rod having a first end separated from a second end by one or more sidewalls, wherein said one or more sidewalls connect said first end to said second end; and
at least one of said one or more sidewalls defines a marking, wherein said marking was formed by removing a portion of said anodization.

21. The apparatus of claim 20, wherein said at least one of said one or more sidewalls includes two sidewalls, each sidewall defining a marking.

22. The apparatus of claim 21, wherein said markings are offset from each other by approximately 180 degrees.

23. The apparatus of claim 20, wherein said at least one of said one or more sidewalls includes six sidewalls that form a hexagonal cross-sectional shape.

24. The apparatus of claim 20, wherein said spinal rod is bent to a sagittal alignment of one or more vertebrae in a medical patient.
